(19) United States
(12) Patent Application Publication
(10) Pub. No.: US 2021/0333278 A1
(43) Pub. Date: Oct. 28, 2021

(54) VIRAL RESPIRATORY INFECTION DETECTION DEVICE

(71) Applicant: Core Diagnostics Corporation, Georgetown, TX (US)

(72) Inventor: Wilma MANGAN, Santa Clara, CA (US)

(21) Appl. No.: 17/242,887

(22) Filed: Apr. 28, 2021

Related U.S. Application Data
(60) Provisional application No. 63/016,735, filed on Apr. 28, 2020.

(51) Int. Cl. G01N 33/569; G01N 33/543; B01L 3/00

(52) U.S. Cl. CPC G01N 33/56983; G01N 33/54386; B01L 3/5023; B01L 2300/0681; B01L 2300/0825; B01L 2400/0406; B01L 2300/161; G01N 2333/165

(57) ABSTRACT

The present disclosure may provide a system and method for using a viral respiratory infection detection device, a method comprising: obtaining a biological sample from a subject; preparing the biological sample for testing; placing at least a portion of the prepared biological sample into a sample port of a viral respiratory infection detection device thereby contacting a sample pad with the prepared biological sample thus initiating a first test strip and a second test strip, wherein the first testing strip is formed to detect the binding of a respiratory virus to a recombinant human receptor protein, wherein the second testing strip is formed to detect the binding of a specific respiratory virus to a specific recombinant spike glycoprotein; and analyzing the results of the first test strip and the second test strip.

### BACKGROUND

Screening tests are critical tools in dealing with rapidly evolving and large-scale outbreaks like Covid-19. These tests can rapidly identify at-risk patients, allowing the medical community to focus its resources on the patients that need help the most.

Screening assays may be developed and utilized to detect viruses or a specific strain of virus. An example of a screening assay may include a two-site noncompetitive immunoassay. In a two-site noncompetitive immunoassay, a biological sample may be placed on a solid support that may be based on a series of capillary beds. A biological sample can be sourced from mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender. The biological sample may then flow up the solid support via capillary action and contact a first binding agent. The first binding agent may have a specificity for a target analyte (e.g., a virus, a specific strain of virus, a control). In certain embodiments, the first binding agent may also comprise a detectable label. If the target analyte is present in the biological sample, the target analyte will then bind with the first binding agent, thus immobilizing the target analyte to the solid support and forming an analyte-first binding agent complex. The analyte-first binding agent complex may then proceed up the solid support and contact a second binding agent. In certain embodiments, the second binding agent may also have a specificity for the target analyte and may bind to the analyte-first binding agent complex. The second binding agent may capture the detectable label thus forming a visible marker on the solid support. The target analyte may be sandwiched between the first binding complex and the second binding complex. The two-site noncompetitive immunoassay or a plurality of two-site noncompetitive immunoassays may be disposed within a testing device. In certain embodiments, the testing device may be a disposable single use testing device used at home or in a clinic setting.

### SUMMARY

Provided are methods for detecting a viral infection. An example method comprises obtaining a biological sample from a subject; placing at least a portion of the biological sample into a sample port of a viral infection detection device to contact a sample pad with the biological sample and initiate a detection test on a first test strip and also a second test strip, wherein the first test strip detects the binding of a respiratory virus to a recombinant human receptor protein, wherein the second test strip detects the binding of a specific virus to a specific recombinant spike glycoprotein; and analyzing the results of the detection test initiated on the first test strip and the second test strip.

Additionally or alternatively, the method may include one or more of the following features individually or in combination. The viral infection detection device may comprise a body having the sample pad disposed therein; wherein the body comprises a material selected from the group consisting of polyester, polyethylene terephthalate glycol-modified, polyethylene terephthalate, polyethylene terephthalate, polybutylene terephthalate, polytrimethylene terephthalate, polyethylene naphthalate, polycarbonate, polyvinyl chloride, polystyrene, styrene-acrylonitrile resin, acrylonitrile-butadiene-styrene, and any combinations thereof. The first test strip and the second test strip may be formed on solid support configured to transport a fluid through an interconnected series of capillary beds. The solid support may comprise a hydrophobic plastic modified to render a surface of the solid support to be hydrophilic. The hydrophobic solid support may be modified by a plasma etching and corona treatment. The solid support may comprise a material selected from the group consisting of porous paper, microstructured polymers, sintered polymers, and any combination thereof. At least one of the detection tests may be a qualitative lateral flow immunoassay. The qualitative lateral flow immunoassay may be a qualitative lateral flow immunoassay selected from the group consisting of a competitive homogeneous immunoassay, a competitive heterogeneous immunoassay, a one-site noncompetitive immunoassay, a two-site noncompetitive immunoassay, and any combinations thereof. The sample pad may comprise a plurality of stacked membranes used to trap red cells and allow plasma or serum to flow to the specific test capturing biomarker. The recombinant human receptor protein may comprise a recombinant angiotensin-converting enzyme 2 receptor. The recombinant spike glycoprotein may comprise a spike glycoprotein trimer nucleoprotein of a coronavirus. The analyzing the results of the detection test may comprise viewing a marker on at least one of the first test strip or second test strip. The marker may be visible for viewing to a human eye.

Provided are various embodiments of a viral infection detection device. An example viral infection detection device comprises a sample pad comprising a first test strip and also a second test strip, wherein the first test strip detects the binding of a respiratory virus to a recombinant human receptor protein, and wherein the second test strip detects the binding of a specific virus to a specific recombinant spike glycoprotein. The viral infection detection device further comprises a body, wherein the sample is disposed within the body.

Additionally or alternatively, the viral infection detection device may include one or more of the following features individually or in combination. The body may comprise a material selected from the group consisting of polyester, polyethylene terephthalate glycol-modified, polyethylene terephthalate, polyethylene terephthalate, polybutylene terephthalate, polytrimethylene terephthalate, polyethylene naphthalate, polycarbonate, polyvinyl chloride, polystyrene, styrene-acrylonitrile resin, acrylonitrile-butadiene-styrene, and any combinations thereof. The first test strip and the second test strip may be formed on solid support configured to transport a fluid through an interconnected series of capillary beds. The solid support may comprise a hydrophobic plastic modified to render a surface of the solid support to be hydrophilic. The hydrophobic solid support may be modified by a plasma etching and corona treatment. The solid support may comprise a material selected from the group consisting of porous paper, microstructured polymers, sintered polymers, and any combination thereof. At least one of the detection tests may be a qualitative lateral flow immunoassay. The qualitative lateral flow immunoassay may be a qualitative lateral flow immunoassay selected from the group consisting of a competitive homogeneous immunoassay, a competitive heterogeneous immunoassay, a one-site noncompetitive immunoassay, a two-site noncompetitive immunoassay, and any combinations thereof. The sample pad may comprise a plurality of stacked membranes used to trap red cells and allow plasma or serum to flow to the specific test capturing biomarker. The recombinant human receptor protein may comprise a recombinant angiotensin-converting enzyme 2 receptor. The recombinant spike glycoprotein may comprise a spike glycoprotein trimer nucleoprotein of a coronavirus.

### TECHNICAL FIELD

The present disclosure relates to a testing device capable of testing for a viral respiratory infection.

### BRIEF DESCRIPTION OF THE DRAWINGS

FIG. 1 illustrates an embodiment of a viral respiratory infection detection device.

FIG. 2 illustrates an embodiment of a first test strip and a second test strip.

FIG. 3 illustrates an embodiment of the testing strips disposed within the body.

### DETAILED DESCRIPTION

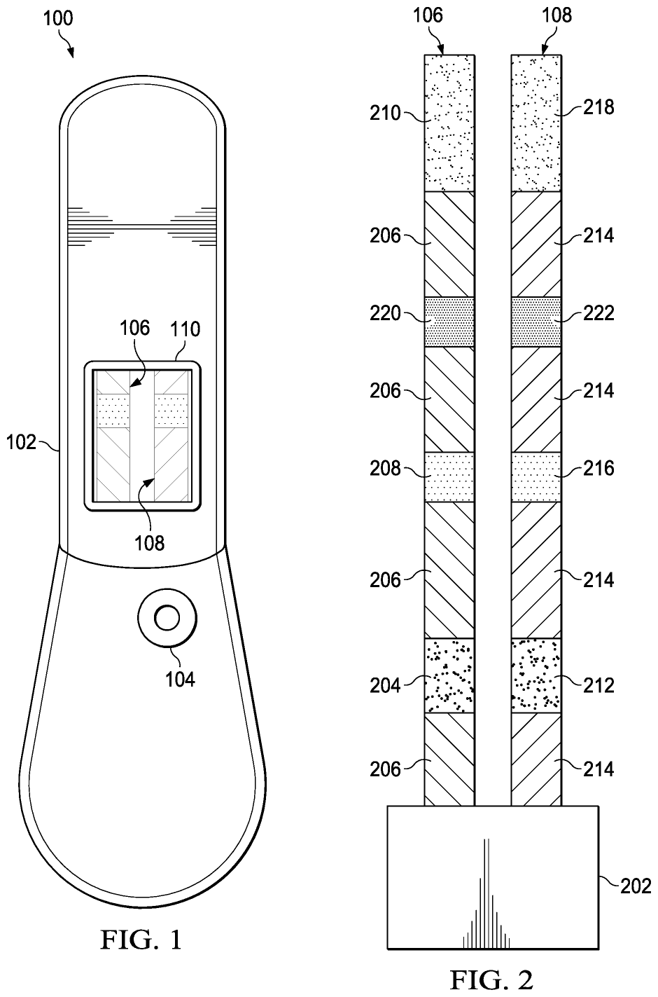

FIG. 1 illustrates an embodiment of a viral respiratory infection detection device 100. The viral respiratory infection detection device 100 may comprise a body 102, a sample port 104, a first test strip 106, a second test strip 108, and a transparent section 110 of the body 102 in which the test results may be displayed.

Body 102 may be any hollow structure capable of fully enclosing first test strip 106 and second test strip 108. Body 102 may be of any suitable size and shape and should not be limited herein. Suitable shapes may include, but are not limited to, rectangle, oval, square, circle, any shape, the like, and/or any combinations thereof. Body 102 may be formed of any suitable material capable of protecting the first test strip 106 and the second test strip 108 from interruptions and deformation. Suitable materials may include, but are not limited to, polymers, elastomers, metals, metal alloys, composite materials, the like, and/or any combinations thereof. The body 102 may be made from a suitable material that may be selected based on its properties including, but not limited to, thermal conductivity, clarity for optical transmission, mechanical properties for easy welding, surface properties that may allow for uniform coating and stability of reagent, neutrality to the liquid medium to prevent interference with the assay, the like, and/or any combinations thereof. Suitable materials may include plastics with high free surface energies and low water sorption. In an embodiment, suitable materials may include, but are not limited to, polyester, polyethylene terephthalate glycol-modified, polyethylene terephthalate, polyethylene terephthalate, polybutylene terephthalate, polytrimethylene terephthalate, polyethylene naphthalate, polycarbonate, polyvinyl chloride, polystyrene, styrene-acrylonitrile resin (SAN), acrylonitrile-butadiene-styrene (ABS), the like, and/or any combinations thereof. In certain embodiments, the solid support may comprise a hydrophobic plastic that may be modified to render the surfaces of hydrophilic. In a non-limiting example, this modification, along with the present disclosure, may be accomplished by any suitable means and should not be limited herein. In an embodiments, the hydrophobic plastic solid support may be modified to comprise a hydrophilic surface via plasma etching and corona treatment. Optionally, a molded solid support may be used. The solid support may be formed or obtained in any suitable manner and should not be limited herein.

The viral respiratory infection detection device 100 may further comprise a sample port 104 disposed within the body 102. The sample port 104 may be of any size and shape capable of receiving a biological sample and contacting the biological sample with a sample pad 202 (referring to FIG. 2). The sample pad 202 may comprise a first test strip 106 and a second test strip 108. Once the sample pad receives the biological sample, the first test strip 106 and the second test strip 108 may be initiated and each assay may begin. Each assay may take approximately 5 minutes to about 25 minutes to complete. Each assay may take approximately about 5 minutes or greater. Optionally, each assay may take approximately about 5 minutes or less.

Referring now to FIG. 2, FIG. 2 illustrates an embodiment of the first test strip 106 and the second test strip 108. The first test strip 106 and the second test strip 108 may be formed such that each test strip 106, 108 may be capable of performing a lateral flow assay. In an embodiment, the first test strip 106 and the second test strip 108 may be formed to perform a qualitative lateral flow immunoassay. Suitable qualitative lateral flow immunoassays may include, but are not limited to, a competitive homogeneous immunoassay, a competitive heterogeneous immunoassay, a one-site noncompetitive immunoassay, a two-site noncompetitive immunoassay (e.g., a sandwich assay), the like, or any combinations thereof. In certain embodiments, the first test strip 106 and the second test strip 108 may perform different assays. Alternatively, the first test strip 106 and the second test strip 108 may perform the same assay. In a non-limiting example, first test strip 106 and second test strip 108 may be formed to perform a two-site noncompetitive immunoassay. In one example of a two-site noncompetitive immunoassay, a biological sample may comprise a target analyte. In certain embodiments, the target analyte may be an antigen. The target analyte may first be bound to a labelled binding agent thereby forming a target analyte labelled binding agent complex. Then the target analyte labelled binding agent complex may be bound to a capture binding agent, such that the target analyte may be “sandwiched” between the labelled binding agent and the capture binding agent. The labelled binding agent and the capture binding agent may be selected to have a specificity for the target analyte. Once the target analyte labelled binding agent complex binds to the capture binding agent, the capture binding agent may detect the labelled binding agent thereby forming a visible marker on the test strip. In a non-limiting example, the visible marker may be a horizontal line spanning the width of the test strip. A visible marker may indicate a positive test result thereby confirming the presence of the target analyte in the biological sample. If the assay is performed and a visible marker is not formed, this may indicate a negative test result. The assay may produce a negative result when the target analyte is not present in the biological sample. The results may then be interpreted by a user.

Each test strip 106, 108 may be formed of any suitable solid support capable of transporting a fluid spontaneously through an interconnected series of capillary beds. In an embodiment, a solid support may be a material based on a series of capillary beds such that the sample may move across the test strip. Suitable solid supports may include, but are not limited to, pieces of porous paper, microstructured polymers, sintered polymers, the like, and/or any combinations thereof. Disposed on the solid support, the test strip 106, 108 may further comprise a sample pad 202, a conjugate pad 204, 212, a transport membrane 206, 214, a test line 208, 216, a control line 220, 222, and an absorbent pad 210, 218.

Each test strip 106, 108 may further comprise a sample pad 202. The biological sample is received on the testing strip at the sample pad 202 location. Any suitable sample pad 202 capable of receiving a biological sample may be used. In a non-limiting example, the sample pad 202 may comprise a permeable matrix of material. In an embodiment, sample pad 202 may comprise an absorbent material or a non-absorbent material. In an embodiment, sample pad 202 may comprise a hydrophilic material. In an embodiment, sample pad 202 may comprise a woven material, a non-woven material, and/or any combinations thereof. In a non-limiting example, suitable materials for the matrix may include, but are not limited to glass fiber, nylon, rayon, cotton, acrylic and polyester and/or any combination of the like.

In certain embodiments, the sample pad 202 may function as a filter to aid the flow or trap interfering agents of a biological sample, adjust parameters of the biological sample, add detergents thereby improving wetting, remove interfering agents found in the biological sample, adding trapping agents, the like, and/or any combinations thereof. Optionally, the sample pad 202 may be used to adjust several parameters of the biological sample to adjust the flow of the sample. Some of the parameters may include but are not limited to, pH, viscosity, the like, and/or any combinations thereof. Optionally, sample pad 202 may function to add detergents thereby improving wetting. Optionally, the sample pad may be used as a filter to remove interfering agents from the biological sample based on size exclusions. The sample pad 202 may comprise a filter or a plurality of filters stacked based on the pore size. Optionally, the filter may trap unbound antibodies, clotting agents, and the like, and/or any combinations thereof. Optionally, the sample pad may be used to add trapping agents such as antibodies, enzymes, precipitating agents, polymers, the like, and/or any combinations thereof to the biological sample.

In an embodiment, sample pad 202 may comprise a plurality of stacked membranes to alter the obtained biological sample. In a non-limiting example, sample pad 202 may be used to trap red cells and allow plasma or serum to flow to the specific test capturing biomarker. Optionally, a two-layer structure may be employed to produce plasma from whole blood, wherein the two-layer structure may include a blood separation layer and filter layer. The blood separation layer and the filter layer may work in tandem to provide for red blood cell free plasma. As used herein, the term “plasma” may refer to a substantially colorless fluid obtained from a whole blood sample after red blood cells have been removed by the separation process.

After the sample pad 106, 108 receives the biological sample, the biological sample may then migrate to a conjugate pad 204, 212. The conjugate pad 204, 212 may comprise a labelled binding agent. As used herein, a “labelled binding agent” may refer to an antibody conjugated with a marker that is specific to the target analyte conjugated to a solid support. Suitable antibodies may include, but are not limited to, recombinant human receptor proteins, recombinant spike glycoprotein of a virus, a polypeptide at least partially encoded by an immunoglobulin gene, a polypeptide at least partially encoded by a plurality of immunoglobulin genes, immunoglobulin genes, fragments of an immunoglobulin gene that may have an affinity for the analyte, fragments of a plurality of immunoglobulin genes that may have an affinity for the analyte, the like, and/or any combinations thereof. As used herein, “antibody” may also include, but is not limited to, a polypeptide substantially encoded by an immunoglobulin gene or immunoglobulin genes, or fragments thereof which specifically bind and recognize the antigen-specific binding region (idiotype) of antibodies produced by the host in response to exposure to trichomonas antigen(s). Examples may include, polyclonal, monoclonal, chimeric, humanized, and single chain antibodies, the like, and/or any combinations thereof. Fragments of immunoglobulins, may include Fab fragments and fragments produced by an expression library, including phage display. In a non-limiting example, a recombinant human receptor may comprise a recombinant angiotensin-converting enzyme 2 (ACE 2) receptor, the like, and/or any combinations thereof. In a non-limiting example, a suitable recombinant spike glycoprotein may comprise a spike glycoprotein trimer nucleoprotein of a coronavirus (e.g., spike glycoprotein subunit 1 or 2 of SARS-CoV-2), the like, and/or any combinations thereof. The labelled binding agent may be conjugated with any marker including, but not limited to, conjugated gold, carbon, colored latex, nanoparticles, magnetic beads, colored polystyrene beads, fluorescent dyes, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), the like, and/or any combinations thereof. As used herein, the terms “marker” and “detectable label” refer to any molecule capable of detection and should not be limited herein.

The conjugate pad 204, 212 may further comprise a conjugate control. As used herein, “conjugate control” may refer to a control sample conjugated with a marker that does not have an affinity for the target analyte. The conjugate control may be used to verify the validity of the assay. The control sample may be conjugated with a marker including, but not limited to, conjugated gold, carbon, colored latex, nanoparticles, magnetic beads, colored polystyrene beads, fluorescent dyes, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), the like, and/or any combinations thereof. As used herein, the terms “marker” and “detectable label” refer to any molecule capable of detection and should not be limited herein.

After contacting the conjugate pad 204, 212, the sample may migrate along the test strip 106, 108 through a transport membrane 206, 214. Any suitable transport membrane 206, 214 capable of aiding in the migration of the sample may be used. Suitable transport membrane 206, 214 may include, but is not limited to, nitrocellulose membrane, nylon, cellulose acetate, other polyesters, other porous membranes, the like, and/or any combinations thereof. The sample may then migrate through the transport membrane 206, 214 to the test line 208, 216. The test line 208, 216 may be disposed atop the transport membrane 206, 214. The test line 208, 216 may comprise a capture binding agent. Any suitable capture binding agent with a specificity for the target analyte may be used and should not be limited herein. Suitable capture binding agents may include, but are not limited to, a primary antibody, an anti-host antibody, a multi-antigenic peptide protein, protein conjugated to ligands, biotin, avidin, streptavidin, haptens, the like, and/or any combinations thereof. As the sample contacts the test line 208, 216, the target analyte labelled binding agent complex binds with the capture binding agent thereby immobilizing the target analyte on the membrane 206, 214. Once bound, the capture binding agent may detect the labelled binding agent thereby forming a visible marker on the test strip 106, 108. In a non-limiting example, the visible marker may be a horizontal line spanning the width of the test strip 106, 108.

The remaining sample may then continue to migrate through the membrane 206, 214 until contacting the control line 220, 222. The control line 220, 222 may comprise an anti-host antibody with a specificity for the control sample. Once the anti-host antibody binds with the control sample conjugated with a marker, the control sample may be immobilized on the transport membrane 206, 214 and a visible marker may be formed on the test strip 106, 108 thereby indicating that this is a valid assay. In a non-limiting example, the visible marker may be a horizontal line spanning the width of the test strip 106,108. Any remaining sample may then migrate through the transport membrane 206, 214 until contacting the absorbent pad 210, 218.

Optionally, the formed marker on the test strip 106, 108 at the test line 208, 216 and the control line 220, 222 may be a fluorescent line not visible to the naked eye without an aid. Once the assay is complete, a filter lens may be placed on an imaging device (e.g., the camera on a mobile phone) thereby allowing the user to see the results of the assay. Optionally, the imaging device may record the results of the assay and wirelessly transmit the data to an off-site information handling system. The off-site information handling system may store and analyze the data, among other things.

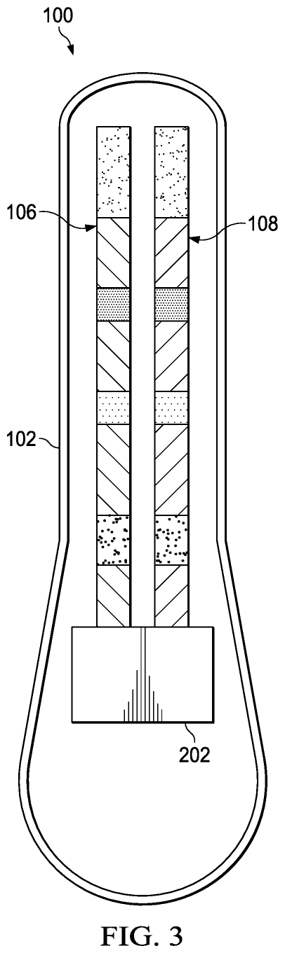

FIG. 3 illustrates an embodiment of a viral respiratory infection detection device 100 with the interior of body 102 visible. In the illustrated embodiment, the sample pad 202, the first test strip 106, and the second test strip 108 are disposed in the interior of the body 102 such that the sample pad 202 is aligned with the sample port 104 (illustrated in FIG. 1) and also such that at least a portion of the first test strip 106 and the second test strip 108 are visible through the viewing window 110 (illustrated in FIG. 1) when the body 102 is closed.

Referring back to FIG. 1, the viral respiratory infection detection device 100 may be a single use disposable dual test for at-home testing or for in-clinic testing. First, the user may obtain a biological sample from a subject. The user may be a health care professional, a human subject, or another human in close proximity to the subject. Any suitable biological sample from a subject of interest may be obtained including, but not limited to, urine, blood, saliva, tissue, the like, and/or any combinations thereof. Biological samples may be collected in any suitable manner including, but not limited to, oropharyngeal swab, nasopharyngeal swab, whole blood collection devices, vacutainers, capillary tubes, urine collection cups, fecal virginal smears, the like, and/or any combinations thereof.

The user must then prepare the collected sample for testing. In a non-limiting example, a collected nasopharyngeal swab may be inserted into a solution and agitated thereby extracting the biological sample from the swab. A portion of the extracted biological sample may then be disposed within a sample port 104 in the viral respiratory infection detection device 100. The biological sample may then flow to a sample pad 202. Connected to the sample pad 202 may be a first test strip 106 and a second test strip 108. In an embodiment, the first test strip 106 may be formed to detect a viral respiratory infection and the second test strip 108 may be formed to detect a specific species of a viral respiratory infection, or vice versa. A viral respiratory infection may include, but is not limited to, coronavirus, influenza virus, respiratory syncytial virus, parainfluenza virus, adenovirus, rhinovirus, human metaphneumovirus, enterovirus, the like, and/or any combinations thereof. A specific species of a viral respiratory infection may include, but is not limited to, MERS-CoV, SARS-CoV, SARS-CoV-2, HCoV-229E, HCoV-NL63, HCoV-OC43, HCoV-HKU1, H1N1, H3N2, H5N1, influenza A, influenza B, influenza C, the like, and/or any combinations thereof.

In an embodiment, the first test strip 106 may be formed to detect the binding of a respiratory virus to a recombinant human receptor. This test may determine if the subject from which the biological sample came from has a viral respiratory infection. The first test strip 106 may comprise a sample pad 202, a first conjugate pad 204, a first transport membrane 206, a first test line 208, a first control line 220, and a first absorbent pad 210. The first conjugate pad 204 comprises a first labelled binding agent, wherein the first labelled binding agent comprises a recombinant human receptor and a control conjugate. The first test line 208 comprises a first capture binding agent, wherein the first capture binding agent comprises an anti-host antibody with a specificity for a respiratory virus. In an embodiment, the first test strip 106 will result in a positive test if a visible marker is present at the first test line 208 and the first control line 220 after the assay verifies testing validity. A positive test may indicate that the human test subject has a respiratory virus. In an embodiment, the first test strip 106 will result in a negative test if there is no visible marker on the first test line 208 and a visible marker is present on the first control line 220. A negative test may indicate that the human subject does not have a respiratory virus. The test may be inconclusive if a visible marker is not present at the first test line 208 or the second test line 216 indicating that there was an error with the assay and an additional test may be required.

The second test strip 108 may be formed to detect the binding of a specific respiratory virus to a recombinant human receptor protein which is bound to an antibody to determine if the subject from which the biological sample came from has a specific viral respiratory infection. The second test strip 108 may comprise a sample pad 202, a second conjugate pad 212, a second transport membrane 214, a second test line 216, a second control line 222, and a second absorbent pad 218. The second conjugate pad 212 comprises a second labelled binding agent, wherein the second labelled binding agent comprises an antibody or a recombinant viral protein and a control conjugate. The second test line 216 comprises a second capture binding agent, wherein the second capture binding agent comprises an anti-host antibody with a specificity for the specific respiratory virus. In an embodiment, the second test strip 108 will result in a positive test if a visible marker is present at the second test line 216 and the second control line 222 after the assay is completed. A positive test may indicate that the human test subject has a specific respiratory virus. In an embodiment, the second test strip 108 will result in a negative test if there is no visible marker on the second test line 216 and a visible marker is present on the second control line 222. A negative test may indicate that the human subject does not have that specific respiratory virus. The test may be inconclusive if a visible marker is not present at the second test line 216 or the second control line 222, thereby indicating that there was an error with the assay and an additional test may be required.

Once both assays are done, the user may then analyze the results of each test strip 106, 108 via the transparent section 110 of the body 102 of the viral respiratory infection detection device 100 adjacent to the sample port 104. For example, if the first test strip 106 is positive and the second test strip 108 is positive, then the user may determine that the human subject is infected with the specific species of viral respiratory infection. If the first test strip 106 is positive and the second test strip 108 is negative, then the user may determine that the human subject is infected with a viral respiratory infection but not the specific species of viral respiratory infection. If the first test strip 106 and the second test strip 108 are negative, then the user may determine that the user does not have a viral respiratory infection. After the user has analyzed the test results, the user may then inform the human subject of the results. The results may allow the human subject to determine if medical care is required. The user may then properly dispose of the single use viral respiratory infection detection device 100. In an embodiment, the first test strip 106 and the second test strip 108 may undergo testing simultaneously. In an embodiment, the first test strip 106 is different from the second test strip 108, wherein the results of each test strip may be used to draw different conclusions regarding the health of the subject.

The test results may be analyzed and/or detected in any suitable manner and should not be limited herein. The test results may be detected and/or analyzed by way of visual inspection, electronic detection, radioactive detection, the like, and/or any combinations thereof. In a non-limiting example, the viral respiratory detection device 100 may comprise a detection system capable of automatically detecting the results. The detection system (not shown) may comprise an excitation source, a monochromator and a detector array. In an embodiment, the monochromator may be any device capable of spectrally resolving light components or a set of narrow band filters. The excitation source may comprise infrared, blue, or UV wave-lengths, wherein the excitation wavelength may be shorter than the emission wavelength(s) to be detected. In an embodiment, the detection system may comprise a broadband UV light source. In a non-limiting example, the broadband UV light source may comprise a deuterium lamp with a filter in front, wherein the output of a white light source such as a xenon lamp or a deuterium lamp after passing through a monochromator to extract out the desired wave-lengths. In a non-limiting example, the broadband UV light source may be a continuous wave gas laser. Suitable continuous wave gas lasers may include, but are not limited to, an argon ion laser (e.g., 457 nm, 488 nm, 514 nm, etc.), a helium cadmium laser, a solid-state diode laser, a solid-state diode laser in the blue, a gallium nitride laser diode, a gallium arsenide semiconductor diode based laser, a yttrium-aluminum garnet laser, a yttrium lithium fluoride based laser, any pulsed laser with an output in the blue, the like, and/or any combinations thereof. In an embodiment, the continuous wave gas laser may comprise a single output, a doubled output, a tripled output, the like, and/or any combinations thereof.

In an embodiment, emitted light from the sample or the reactants in the capture zone may be detected with an imaging device (not shown) that may provide spectral information for the color change. Suitable imaging devices may include, but are not limited to, a grating spectrometer, prism spectrometer, imaging spectrometer, the like, use of interference (bandpass) filters, and/or any combinations thereof. Optionally, a two-dimensional area imager such as a charge-coupled device camera may be used to detect and image more than one object simultaneously. Spectral information may be generated by collecting more than one image via different bandpass, long pass, or short pass filters (e.g., interference filters, electronically tunable filters etc.). Optionally, more than one imager may be used to gather data simultaneously through dedicated filters. In a non-limiting example, the filter may be changed in front of a single imager. Imaging based systems, like the Biometric Imaging system, may scan a surface to find fluorescent signals.

The figures (Figs.) and the above description may relate to embodiments of the present disclosure by way of illustration only. It may be noted that from the above disclosure, alternative embodiments of the structures and methods disclosed herein may be readily recognized as viable alternatives that may be employed without departing from the principles of what is claimed.

The figures depict embodiments of the disclosed system (or method) for purposes of illustration only. One skilled in the art, along with the present disclosure, may recognize that alternative embodiments of the structures and methods illustrated herein may be employed without departing from the principles described herein.

As used herein any reference to “one embodiment,” “an embodiment,” or “some embodiments” means that a particular element, feature, structure, or characteristic described in connection with the embodiment may be included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article or apparatus that comprises a list of elements may not necessarily be limited to only those elements but may include other elements not expressly listed or inherent to such process, method, article or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This may be done merely for convenience and to give a general sense of the disclosure. This description may be read to include one and/or at least one and the singular also includes the plural unless it is obvious that it may be meant other-wise.

1. A method for detecting a viral infection, the method comprising:
obtaining a biological sample from a subject;
placing at least a portion of the biological sample into a sample port of a viral infection detection device to contact a sample pad with the biological sample and initiate a detection test on a first test strip and also a second test strip, wherein the first test strip detects the binding of a respiratory virus to a recombinant human receptor protein, wherein the second test strip detects the binding of a specific virus to a specific recombinant spike glycoprotein; and
analyzing the results of the detection test initiated on the first test strip and the second test strip.

2. The method of claim 1, wherein the viral infection detection device comprises a body having the sample pad disposed therein; wherein the body comprises a material selected from the group consisting of polyester, polyethylene terephthalate glycol-modified, polyethylene terephthalate, polyethylene terephthalate, polybutylene terephthalate, polytrimethylene terephthalate, polyethylene naphthalate, polycarbonate, polyvinyl chloride, polystyrene, styrene-acrylonitrile resin, acrylonitrile-butadiene-styrene, and any combinations thereof.

3. The method of claim 1, wherein the first test strip and the second test strip are formed on solid support configured to transport a fluid through an interconnected series of capillary beds.

4. The method of claim 3, wherein the solid support comprises a hydrophobic plastic modified to render a surface of the solid support to be hydrophilic.

5. The method of claim 4, wherein the hydrophobic solid support is modified by a plasma etching and corona treatment.

6. The method of claim 3, wherein the solid support comprises a material selected from the group consisting of porous paper, microstructured polymers, sintered polymers, and any combination thereof.

7. The method of claim 1, wherein at least one of the detection tests is a qualitative lateral flow immunoassay.

8. The method of claim 7, wherein the qualitative lateral flow immunoassay is a qualitative lateral flow immunoassay selected from the group consisting of a competitive homogeneous immunoassay, a competitive heterogeneous immunoassay, a one-site noncompetitive immunoassay, a two-site noncompetitive immunoassay, and any combinations thereof.

9. The method of claim 1, wherein the sample pad comprises a plurality of stacked membranes used to trap red cells and allow plasma or serum to flow to the specific test capturing biomarker.

10. The method of claim 1, wherein the recombinant human receptor protein comprises a recombinant angiotensin-converting enzyme 2 receptor.

11. The method of claim 1, wherein the recombinant spike glycoprotein comprises a spike glycoprotein trimer nucleoprotein of a coronavirus.

12. The method of claim 1, wherein the analyzing the results of the detection test comprises viewing a marker on at least one of the first test strip or second test strip.

13. The method of claim 1, wherein the marker is visible for viewing to a human eye.

14. A viral infection detection device comprising:
a sample pad comprising a first test strip and also a second test strip, wherein the first test strip detects the binding of a respiratory virus to a recombinant human receptor protein, and wherein the second test strip detects the binding of a specific virus to a specific recombinant spike glycoprotein; and
a body, wherein the sample is disposed within the body.

15. The viral infection detection device of claim 14, wherein the body comprises a material selected from the group consisting of polyester, polyethylene terephthalate glycol-modified, polyethylene terephthalate, polyethylene terephthalate, polybutylene terephthalate, polytrimethylene terephthalate, polyethylene naphthalate, polycarbonate, polyvinyl chloride, polystyrene, styrene-acrylonitrile resin, acrylonitrile-butadiene-styrene, and any combinations thereof.

16. The viral infection detection device of claim 14, wherein the first test strip and the second test strip are formed on solid support configured to transport a fluid through an interconnected series of capillary beds.

17. The viral infection detection device of claim 16, wherein the solid support comprises a hydrophobic plastic modified to render a surface of the solid support to be hydrophilic.

18. The viral infection detection device of claim 14, wherein the sample pad comprises a plurality of stacked membranes used to trap red cells and allow plasma or serum to flow to the specific test capturing biomarker.

19. The viral infection detection device of claim 14, wherein the recombinant human receptor protein comprises a recombinant angiotensin-converting enzyme 2 receptor.

20. The viral infection detection device of claim 14, wherein the recombinant spike glycoprotein comprises a spike glycoprotein trimer nucleoprotein of a coronavirus.
